(19) United States
(12) Patent Application Publication
(10) Pub. No.: US 2013/0040325 A1
(43) Pub. Date: Feb. 14, 2013

(54) Enzyme Linked Immunosorbent Assay (ELISA) Method and Kit for Detecting Soluble Programmed Cell Death Protein 5 (PDCD5)

(71) Applicants: Yingyu Chen, Beijing (CN); Dalong Ma, Beijing (CN); Quansheng Song, Beijing (CN); Yingmei Zhang, Beijing (CN); Huan Pan, Beijing (CN); Lanjun Xu, Beijing (CN)

(72) Inventors: Yingyu Chen, Beijing (CN); Dalong Ma, Beijing (CN); Quansheng Song, Beijing (CN); Yingmei Zhang, Beijing (CN); Huan Pan, Beijing (CN); Lanjun Xu, Beijing (CN)

(73) Assignee: PEKING UNIVERSITY, Beijing (CN)

(21) Appl. No.: 13/521,087

(22) PCT Filed: Jan. 17, 2011
(86) PCT No.: PCT/CN2011/000066

§ 371 Date: Sep. 18, 2012

(30) Foreign Application Priority Data
Jan. 21, 2010 (CN) 201010034529.7

(51) Int. Cl. G01N 33/566; G01N 21/64

(52) U.S. Cl. 435/7.94

(57) ABSTRACT

An enzyme linked immunosorbent assay (ELISA) method and kit for detecting soluble programmed cell death 5 (PDCD5) protein are provided. The method includes the following steps: (1) contacting a sample to be tested with a solid carrier loaded with a first antibody of PDCD5 protein; (2) adding a second antibody of PDCD5 protein, which is capable to binding to a detecting label; (3) adding the detecting label and detecting the bound detecting label. The soluble PDCD5 in a biological sample from a mammal comprising human can be detected by the ELISA method and kit. The method and kit can be used for detecting the level of PDCD5 protein in plasma, serum, urine, cerebrospinal fluid and synovial fluid so that an auxiliary detection method, for diagnosis of autoimmune disease, inflammation (such as hepatitis), and tumor etc., determination of disease course, observation of therapeutic effect and prognosis and medical guidance, is provided.

### TECHNICAL FIELD

The present invention belongs to the field of immunology and biological technology, and in particular, the present invention is related to an ELISA method and kit for detecting soluble PDCD5 protein. The method and kit may be used to detect the level of PDCD5 protein in the body fluid samples obtained from patients with autoimmune disease, various types of inflammation, and tumor, etc., to detect PDCD5 protein in various types of biological samples (such as the supernatant of a cell culture or cell lysate) in the fundamental research, and to detect the PDCD5 protein in biological samples originated from animal models.

### BACKGROUND ART

TFAR19 (TF-1 cell apoptosis-related gene 19) is a new regulating gene for programmed cell death, which was named as “PDCD5” (programmed cell death 5) by Human Gene Nomenclature Committee (HGNC) in 2002. The gene is evolutionarily conserved and widely expressed. The PDCD5 protein consists of 125 amino acids, and is mainly located within cytoplasm and nucleus. Functional studies demonstrated that the PDCD5 is able to promote the apoptosis of various types of tumor cells and it is a potential new cancer suppressor gene, and the effective mechanism of which plays a role in promoting the apoptosis of tumor cells by binding histone acetyltransferase Tip 60 (Chinese Patent No.: 98101869.6; Biochem Biophy Res Comm, 1999, 254:203-210; Neoplasia, 2009, 10(4): 345-354).

Up till now, by using different technologies, a variety of labs at home and abroad have discovered that the abnormal expression of PDCD5 is related to the occurrence and development of many diseases. It is revealed that the expression of PDCD5 is obviously lower in tumor cells than in normal cells, in a lot of research reports, such as liver cancer (Proc. Natl. Acad. Sci. USA, 2001, 98(26):15089-15094; World Chinese Journal of Digestology, 2008, 16(16): 1820-1824; Chinese Journal of General Surgery, 2008, 17(7): 721-724), breast cancer (N. Engl. J. Med, 2001, 344(8):539-548; Proc. Natl. Acad. Sci. USA, 2004, 101 (52): 18147-18152), epithelial ovarian cancer (Chinese Journal of Clinical Obstetrics and Gynecology, 2002, 3(3):164-167), oral squamous cell carcinoma (Acta Scientiarum Naturalium Universitatis Pekinensis (Medical edition), 2005, 37(4):429-432), lung cancer (J. Clin. Oncol., 2006, 24(11):1672-1678), stomach cancer (Apoptosis, 2006, 11(6): 993-1001), multiple myeloma (Modern Medicine & Health, 2007 (21):3184-3187), renal clear cell carcinoma (J. South Med. Univ., 2006, 26 (6): 805-809; 2006, 26 (9): 1316-1318) and brain glioma (Oncol Rep. 2008, 20(3): 573-579; Chinese Journal of Clinical Neurosurgery, 2008, 13(10):611-613), et, al. Among these research reports, the expression of PDCD5 is closely related to the FIGO periodization, histological grade and pathological type of epithelial ovarian cancer. The expression of PDCD5 is down-regulated with the increase of FIGO periodization and histological grade; the low expression level of PDCD5 is also related to the poor prognosis of stomach cancer and renal clear cell carcinoma. Also it has been proved that the expression level of PDCD5 protein is lower in patients with acute myeloid leukemia (AML) than in patients with chronic myeloid leukemia (CML); moreover, in CML patients, particularly CML patients in the acceleration/blastic phase, the expression level of PDCD 5 is negatively correlated with the expression level of BCR-ABL (Acta Scientiarum Naturalium Universitatis Pekinensis (Medical edition), 2002, 34 (6): 676-679; Leuk Res. 2006, 30(9): 1159-1165).

Moreover, the abnormal expression of PDCD5 is also involved in the progresses of some autoimmune diseases and inflammation, such as systemic lupus erythematosus (SLE) (Chinese Journal of Pathophysiology, 2003, 19 (2): 189-193; Chinese Journal of Rheumatology, 2002, 6(5): 328-330), lupus nephritis (Journal of Clinical Pediatrics, 2003, 21 (10): 607-609), psoriasis (Chinese Journal of Dermatology, 2004, 37: 215-217), osteoarthritis (Acta Scientiarum Naturalium Universitatis Pekinensis (Medical edition), 2003, 35(5): 481-484), rheumatoid arthritis (APOPTOSIS, 2007; 12(8): 1433-1441; Chinese Journal of Biochemistry and Molecular Biology 2008, 24(6): 563-568; J. Clin. Rehabil. Tissue Eng. Res., 2008, (24):4667-4671), senile cataract (Chinese Ophthalmic Research, 2002, 20 (6): 514-516), polycystic ovary syndrome (PCOS) (Acta Scientiarum Naturalium Universitatis Pekinensis (Medical edition), 2005, 37 (5): 476-479), and chronic heart failure (National Medical Journal of China, 2005, 85 (10): 676-678), and the like.

The above studies mainly focus on detecting the expression levels of PDCD5 mRNA and protein of PDCD5 in cells of tissue of the healthy person or in the disease state by immunohistochemistry, immunofluorescence and RT-PCR technology, or detecting the level of PDCD5 autoantibody in the blood by using indirect Enzyme-linked immunosorbent assay (ELISA). Among these technologies above, the indirect Enzyme-linked immunosorbent assay is carried out by the following steps: absorbing the PDCD5 on an ELISA plate, adding serum sample from a patient, and then adding IgG against human labeled with horse radish peroxidase, and finally adding the enzyme substrate to allow color development. The level of PDCD5 autoantibody is determined by shade of the color. This method is only capable of detecting the PDCD5 antibody, and is generally applied at a late stage of a disease, and thus the method is not accurately indicative of the level of PDCD5 protein in the body for the occurrence of antigens is earlier than the production of antibodies. Therefore, this method may only be used to detect the PDCD5 antibody, and can not detect the PDCD5 antigen.

Recently, Japanese researchers have found in the study on Toxoplasma gondii PDCD5 that there is a secreted form of Toxoplasma gondii PDCD5, which plays an important role in the apoptosis of the host cell infected by Toxoplasma gondii (Mol Biochem Parasitol. 2008; 159(2):112-120; J. Vet. Med. Sci. 2009 September; 71(9): 1183-1189). It is not yet reported at home and abroad whether there is a secret form of human PDCD5. Considering the abnormal expression of PDCD5 in the cells of tissue of patients with tumor, autoimmune diseases and inflammation, etc., it is necessary to develop a kit which is capable of detecting the soluble PDCD5 proteins in laboratorial or clinical biological samples, thereby it is hopeful for using the soluble PDCD5 protein as a new biomarker, so as to provide an auxiliary detection method, for diagnosis of a disease, determination of disease course, observation of therapeutic effect, medical guidance and prognosis; and to lay the foundation for further exploring the function of PDCD5 protein.

### SUMMARY OF THE INVENTION

The object of the inventions is to provide an antibody sandwich ELISA method for detecting soluble PDCD5 protein, and experimental results show that the method is able to detect the soluble PDCD5 protein in the serum, plasma, urine, pleural-peritonreal effusion, synovial fluid, cerebrospinal fluid and amniotic fluid of clinical patients, and may also be employed to detect the soluble PDCD5 protein in various biological samples in fundamental researches (such as supernatants of cell culture or cell lysates), and is used to detect the soluble PDCD5 protein in biological samples from animal models.

A further object of the invention is to provide an antibody sandwich ELISA kit for detecting soluble PDCD5 protein. The ELISA kit produced in the invention provides a powerful tool for the fundamental researches and clinical studies of PDCD5.

The technical solution for realizing the above objects is as follows:

An ELISA method for detecting soluble PDCD5 protein, which comprises the following steps:

(1) Contacting a sample to be tested with a solid carrier loaded with a first antibody of PDCD5 protein;
(2) Adding a second antibody of PDCD5 protein, which is capable of binding to a detecting label;
(3) Adding the detecting label and detecting the bound detecting label.

In the above method, the sample to be tested may be serum, plasma, urine, pleural-peritoneal effusion, synovial fluid, cerebrospinal fluid, amniotic fluid, supernatant of a cell culture or cell lysate.

Preferably, the method comprises the following steps: repeating steps (1) to (3) except for replacing a series of solutions comprising soluble PDCD5 protein with known concentrations with the sample to be tested, and plotting standard curves; repeating steps (1) to (3) with sample to be tested; determining the concentration of soluble PDCD5 protein in the sample to be tested in accordance with the standard curves.

Preferably, in the above methods, the first antibody of PDCD5 protein is a monoclonal antibody of PDCD5 protein, such as a mouse monoclonal antibody against human PDCD5 protein.

Preferably, in the above methods, the second antibody of PDCD5 protein is a polyclonal antibody of PDCD5 protein, such as a rabbit polyclonal antibody against human PDCD5 protein.

In the above methods, the detecting label may be enzyme, fluoresce or isotope.

The invention further provides an ELISA kit for detecting soluble PDCD5 protein, which comprises:

a first antibody of PDCD5 protein for capturing the soluble PDCD5 protein in the sample to be tested;
a second antibody of PDCD5 protein which is capable of binding to a detecting label;
a series of solutions of soluble PDCD5 protein comprising known concentrations;
a detecting label;
a tool for detecting the detecting label.

Preferably, in the ELISA kit described above, the first antibody of PDCD5 protein is a monoclonal antibody of PDCD5 protein, such as a mouse monoclonal antibody against human PDCD5 protein.

Preferably, in the ELISA kit described above, the second antibody of PDCD5 protein is a polyclonal antibody of PDCD5 protein, such as a rabbit polyclonal antibody against human PDCD5 protein.

Preferably, in the ELISA kit described above, the detecting label may be enzyme, fluoresce or isotope. The tool for detecting the detecting label may be a tool for detecting enzyme, fluoresce or isotope.

Particularly, the ELISA method of the invention for detecting soluble PDCD5 protein comprises the following steps:

(1) Collecting various biological samples, including body fluid samples of various patients or healthy human, supernatants of cell culture or cell lysates.
(2) Dilution and loading of standard: PDCD5 protein standard is set in 16 wells on a plate coated with mouse monoclonal antibody against human PDCD5 protein, and the standard is diluted from 200 ng/ml into 8 serial dilutions: 200 ng/ml, 100 ng/ml, 50 ng/ml, 25 ng/ml, 12.5 ng/ml, 6.25 ng/ml, 3.125 ng/ml, 1.56 ng/ml, and each of the standard is set in 2 duplicate wells at a volume of 0.1 ml/well, and the plate is incubated at 37° C. for 45-60 minutes.
(3) Adding the sample to be tested: the sample to be tested is added into a plate coated with mouse monoclonal antibody against human PDCD5 protein at 0.1 ml/well, and blank control is set at the same time (the blank control well has no sample while the other procedures are identical), and the plate is incubated at 37° C. for 45-60 minutes.
(4) Washing: Liquid in the wells of the plates above is removed and the plate is died by spinning, and then 0.2 ml of washing solution is added into each well for washing, and then repeat the washing for 3 times, and pat the plate dry.
(5) Adding PDCD5 polyclonal antibody: Each well is added with rabbit polyclonal antibody against human PDCD5 which is diluted with sample diluting solution at 0.1 ml/well, and then the plate is incubated at 37° C. for 45-60 minutes.
(6) Washing: Liquid is removed and the plate is died by spinning, and then 0.2 ml of washing solution is added into each well for washing, and then repeat the washing for 3 times, and pat the plate dry.
(7) Adding an anti-rabbit antibody labeled with horse radish peroxidase (HRP): adding the enzyme labeled secondary antibody (0.2-0.5 μL/ml) which is diluted with sample diluting solution, and the plate is incubated at 37° C. for 45-60 minutes.
(8) Washing: Liquid is removed and the plate is died by spinning, and then 0.2 ml of washing solution is added into each well for washing, and then repeat the washing for 3 times, and pat the plate dry.
(9) Coloration: 3,3′5,5′-Tetramethylbenzidine (TMB) coloration solution is added at 0.1 ml/well, and the plated is gently shaken for mixing up, and then colored at 37° C. away from light for 10-15 minutes.
(10) Termination: 50 μl of termination solution is added into each well to terminate the reactions.
(11) Assay: Using the blank well as calibration base line, the optical density (OD value) in each well is measured in sequence at a wavelength of 450 nm. The assay should be carried out within 15 minutes after the termination solution is added.
(12) Plotting the standard curve and calculating the concentration. The standard curve is obtained with the OD values as the horizontal ordinate and the concentration of the standard as the vertical coordinate, and a regression equation is obtained. The OD values of sample to be tested are substituted into the equation to get the concentration of the sample to be tested.

The basis of the antibody sandwich ELISA method in the invention is the immobilization of antibodies and enzyme labeling of antibodies, i.e., the antibody immobilized on the surface of a solid phase carrier retains its immune activity, and the enzyme labeled antibody retains both the immune activity of the antibody and the activity of the enzyme. As an example, the sample to be tested reacts with the antibody immobilized on the surface of the solid phase carrier. Washing procedures are used to separate the antigen-antibody complex formed on the solid phase carrier with other substances in the liquid. Then an antibody labeled with enzyme is added and also bound to the solid phase carrier by way of reaction. After adding a substrate of the enzyme reaction, the substrate is converted into colored products by the catalysis of enzymes. The amount of the product is directly related with the amount of the tested substance in the sample, and thereby a qualitative or quantitative assay is conducted based on the shade of the color. The enzyme possesses high catalytic efficiency, therefore the result of the immune reaction is magnified indirectly, and thus the assay method has very high sensitivity. Furthermore, the antibody sandwich ELISA method may be used for the detecting of a trace of soluble PDCD5 protein antigen.

Based on the above studies, in the current invention the soluble PDCD5 protein in biological samples of mammals, including human is successively detected by use of the above ELISA method and kit. The detecting results of PDCD5 protein expression in the serum of various types of patients and healthy human indicate that: the level of PDCD5 protein in the serum of patients with multiple sclerosis is over 2.5 times higher than that of in the serum of healthy human; the level of PDCD5 protein in the serum of patients with hepatitis is over 4 times higher than that of in the serum of healthy human; the level of PDCD5 protein in the serum of patients with Influenza A is over 4 times higher than that of in the serum of healthy human; the level of PDCD5 protein in the serum of patients with myeloma and chronic myelogenous leukemia are slightly lower than that of in the serum of healthy human, and the difference has significance (p<0.05); for cancer patients which are given radiotherapy the level of PDCD5 in the serum is significantly higher than that of healthy human, and the difference has significance (p<0.0001); for patients with rheumatoid arthritis the level of PDCD5 in the serum is significantly higher than that of healthy human, and the difference has significance (p<0.001); furthermore, in the synovial fluid of patients with rheumatoid arthritis, a significantly higher level of soluble PDCD5 protein is detected compared to that of healthy human, and the difference has significance (p<0.001); the level of PDCD5 protein in the serum of patients with osteoarthritis is not significantly different from that of in the serum of healthy human, but is significantly lower than that of in the serum of patients with rheumatoid arthritis, and the difference has significance (p<0.001) compared to the patients with rheumatoid arthritis; at the same time, the level of PDCD5 protein in the synovial fluid of patients with osteoarthritis is significantly lower than that of in the synovial fluid of patients with rheumatoid arthritis, and the difference has significance (p<0.00001); the level of PDCD5 protein in the serum of patients with systemic lupus erythematosus is higher than that of in the serum of healthy human, and the difference has significance (p<0.05); when the above method and kit are applied in the detection of the level of PDCD5 protein in plasma, serum, urine, cerebrospinal fluid and synovial fluid, they provide a new auxiliary detection method for the diagnosis of autoimmune disease, various inflammations (such as hepatitis), and tumor etc., determination of disease course, observation of therapeutic effect, medical guidance and prognosis. The detecting method and kit are easy in sampling, simple in operation, has higher sensitivity and accuracy, and are thus they are more advantageous in spreading and application.

### BRIEF DESCRIPTION OF THE DRAWINGS

FIG. 1 shows the reactivity of rabbit polyclonal antibody against human PDCD5 protein with PDCD5 protein detected by Western Blot, 1: PDCD5₁₋₁₂₅; 2: PDCD5₂₇₋₁₂₅; 3: PDCD5₂₀₋₁₀₄; 4: PDCD5₃₄₋₁₀₄;

FIG. 2 shows the reactivity of mouse monoclonal antibody against human PDCD5 protein with PDCD5 protein detected by Western Blot, 1: PDCD5₁₋₁₂₅; 2: PDCD5₃₄₋₁₂₅; 3: PDCD5₂₀₋₁₂₅; 4: PDCD5₂₇₋₁₂₅; 5: PDCD5₁₋₁₁₂;

FIG. 3 shows the result of the detection of PDCD5 protein by using the antibody sandwich ELISA method;

FIG. 4 shows the result of the detection of PDCD5 protein in the serum of healthy human and patients with multiple sclerosis by using the antibody sandwich ELISA method;

FIG. 5 shows the result of the detection of PDCD5 protein in the serum of healthy human and patients with hepatitis by using the antibody sandwich ELISA method;

FIG. 6 shows the result of the detection of PDCD5 protein in the serum of healthy human and patients with Influenza A by using the antibody sandwich ELISA method;

FIG. 7 shows the result of the detection of PDCD5 protein in the serum and synovial fluid of healthy human, patients with rheumatoid arthritis and patients with osteoarthritis by using the antibody sandwich ELISA method;

FIG. 8 shows the result of the detection of PDCD5 protein in the serum of healthy human and patients with systemic lupus erythematosus by using the antibody sandwich ELISA method;

FIG. 9 shows the result of the detection of PDCD5 protein in the serum of healthy human and cancer patients after receiving radiotherapy by using the antibody sandwich ELISA method.

### BEST MODE FOR CARRYING OUT THE INVENTION

The invention is further described below with reference to the specific embodiments and the embodiments are provided to illustrate the invention instead of limiting the scope thereof.

### Example 1

### Preparation of Rabbit Polyclonal Antibody Against Human PDCD5₃₄₋₁₂₅ Protein

### Step 1: Immunization of the Animals

100 μg/ml of purified recombinant PDCD5₃₄₋₁₂₅ protein (Archives of Biochemistry and Biophysics, 486 (2): 141-149) is mixed with identical amount of Complete Freund's Adjuvant (produced by Sigma), sufficiently emulsified and then used to immune the rabbit with multi-site injective method in the back of the rabbit. After 2 weeks the immunization is repeated with identical amount (100 μg/ml) of purified recombinant PDCD5₃₄₋₁₂₅ protein plus Complete Freund's Adjuvant (produced by Sigma). Thereafter the immunization is repeated every two weeks (with 100 μg/ml of purified recombinant PDCD5₃₄₋₁₂₅ protein+identical amount of Complete Freund's Adjuvant (produced by Sigma)) and the immunization is carried out for 3 times in total. 7 days after the last immunization, the blood is taken from ear vein, and the titer of the rabbit polyclonal antibody against human PDCD5₃₄₋₁₂₅ in the serum is detected by indirect ELISA. Then the blood is taken from the heart, and the serum is collected and kept under −20° C.

### Step 2: Purification of the Rabbit Polyclonal Antibody Against Human PDCD5₃₄₋₁₂₅ Protein

The titer of the rabbit polyclonal antibody against human PDCD5₃₄₋₁₂₅ protein in the serum of rabbit is 5×10⁵ as detected by indirect ELISA method. The serum is then used to purify the rabbit polyclonal antibody against human PDCD5₃₄₋₁₂₅ protein by using an affinity chromatography method (following the instructions) with HiTrap™ Protein G (produced by Amersham Pharmacia Biotech company). The purified antibody is then undergone SDS-PAGE electrophoresis, and the electrophoresis is operated with conventional procedures (the concentration of the separation gel is 12.5% and the concentration of the stacking gel is 4.5%). The purity of the antibody detected is over 90%, and the purified antibody is kept under −30° C. before use.

Step 3: Reactivity of the Rabbit Polyclonal Antibody Against Human PDCD5₃₄₋₁₂₅ Protein and the PDCD5 protein.

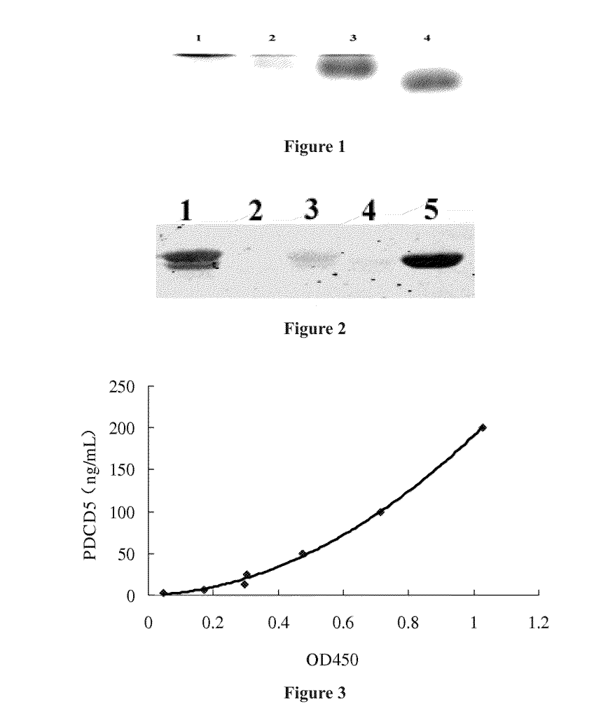

Western Blot immunoblotting assay is employed to detect the binding reaction of the rabbit polyclonal antibody against human PDCD5₃₄₋₁₂₅ protein and the PDCD5 protein. The purified recombinant PDCD5 protein (PDCD5₁₋₁₂₅, PDCD5₂₇₋₁₂₅, PDCD5₂₀₋₁₀₄, PDCD5₃₄₋₁₂₅) is undergone 15% SDS-PAGE electrophoresis and then the protein band in the gel is transferred onto a nitrocellulose membrane in a Bio-Rad electrotransfer system (produced by Schleicher & Schuell company) with conventional method, and then the membrane is blocked with 5% of skim milk powder under 4° C., and then the rabbit polyclonal antibody against human PDCD5₃₄₋₁₂₅ protein is added, and the mixture is reacted under room temperature for 1 hour, and then the membrane is washed for 3 times, each time for 5-10 minutes. Then the membrane is incubated with anti-rabbit IgG fluorescent antibody labeled with Alexa Fluor 780 under room temperature in dark for 1 hour. Then the membrane is washed for 3 times, each time for 5-10 minutes. The fluorophore immobilized on the membrane may be excited by infrared ray, and when interacting an exciting light of 780 nm, the fluorophore gives an emitting light with a wavelength of 820 nm which may be detected by the signal detector of a LI-COR infrared imaging system, Odyssey, Lincoln, Nebr., and the signal obtained may be analyzed using a software provided by Odyssey company. The result is shown in FIG. 1. There is a special reaction between the rabbit polyclonal antibody against human PDCD5₃₄₋₁₂₅ protein and PDCD5₃₄₋₁₂₅ peptide fragment, which indicates that the polyclonal antibody is capable of recognizing the PDCD5 protein with an amino acid sequence from site 34 and thereafter.

### Example 2

### Reactivity of the Mouse Monoclonal Antibody Against Human PDCD5 and the PDCD Protein

The preparation of the mouse monoclonal antibody against human PDCD5 is described as the literature (Acta Acad. Med. Sin., 2000, 22(6):502-504). Western blot immunoblotting assay is employed to detect the binding reaction of the mouse monoclonal antibody against human PDCD5 and the PDCD protein: the reaction steps are identical to step 3 in Example 1. The results are shown in FIG. 2. There is special reaction between the mouse monoclonal antibody against human PDCD5 and the N-terminal of PDCD5 peptide, while there is no special reaction between the mouse monoclonal antibody against human PDCD5 and PDCD5₃₄₋₁₂₅ fragment.

### Example 3

### Establishment of Double-Antibody Sandwich ELISA Method

1. Coating: the mouse monoclonal antibody against human PDCD5 is diluted to a concentration of 1 μg/ml with a pH 9.0, 0.5 M carbonate buffer, and then the antibody is added into a 96-well ELISA plate at 0.1 ml/well, and then the antibody is reacted under 4° C. for 24 hours.
2. Washing: Remove the liquid, and then the plate is dried by spinning 0.2 ml PBS washing solution containing 0.05% Tween 20 is added into each well for washing, and the washing is repeated for 3 times, and then pat the plate dry.
3. Blocking. PBS containing 0.05% Tween 20 and 3% bovine serum albumin (BSA) is added into the coated ELISA plate at 0.15 ml/well, then reacted under 37° C. for 2 hours, or blocked overnight.
4. Washing: Remove the liquid, and then the plate is dried by spinning 0.2 ml PBS washing solution containing 0.05% Tween 20 is added into each well for washing, and the washing is repeated for 3 times, and then pat the plate dry.
5. Dilution and Loading of the Standard: PDCD5 protein standard is set in 16 wells on a plate coated with mouse monoclonal antibody against human PDCD5, and the standard is diluted from 200 ng/ml into 8 serial dilutions: 200 ng/ml, 100 ng/ml, 50 ng/ml, 25 ng/ml, 12.5 ng/ml, 6.25 ng/ml, 3.125 ng/ml, 1.56 ng/ml, and each of the standard is set in 2 duplicate wells at a volume of 0.1 ml/well, and the plate is incubated at 37° C. for 45-60 minutes.
6. Adding the sample to be tested: a plate coated with mouse monoclonal antibody against human PDCD5 protein, 0.1 ml/well, and blank control is set at the same time (the blank control well has no sample while the other procedures are identical), and the plate is incubated at 37° C. for 45-60 minutes.
7. Washing: Remove the liquid, and the plate is dried by spinning, and then 0.2 ml of PBS washing solution containing 0.05% Tween 20 is added into each well for washing, and the washing is repeated for 3 times, and then pat the plate dry.
8. Adding PDCD5₃₄₋₁₂₅ polyclonal antibody: rabbit polyclonal antibody against human PDCD5₃₄₋₁₂₅ (1 μg/ml) diluted with the sample dilution solution (PBS, 0.15 mol/L of phosphate buffer, pH 7.4; the formulation: KH₂PO₄ 0.2 g, Na₂HPO₄ 12H₂O 2.9 g, KCl 0.2 g, NaCl 8.0 g, supplemented with distilled water to 1000 mL) is added at 0.1 ml/well, and then incubated under 37° C. for 45-60 minutes.
9. Washing: Remove the liquid, and the plate is dried by spinning, and then 0.2 ml of PBS washing solution containing 0.05% Tween 20 is added into each well for washing, and the washing is repeated for 3 times, and then pat the plate dry.
10. Adding an anti-rabbit secondary antibody labeled with horse radish peroxidase (HRP): adding the enzyme labeled secondary antibody (0.2-0.5 μL/ml) which is diluted with sample diluting solution, and the plate is incubated at 37° C. for 45-60 minutes.
11. Remove the liquid, and the plate is dried by spinning, and then 0.2 ml of PBS washing solution containing 0.05% Tween 20 is added into each well for washing, and the washing is repeated for 3 times, and then pat the plate dry.
12. Coloration: 3,3′,5,5′-Tetramethylbenzidine (TMB) coloration solution is added at 0.1 ml/well, and the plated is gently shaken for mixing up, and then kept at 37° C. for 10-15 minutes for coloration.
13. Termination: 50 μl of termination solution is added into each well to terminate the reactions.
14. Assay: Using the blank well as calibration base line, the optical density (OD value) in each well is measured in sequence at a wavelength of 450 nm. The assay should be carried out within 15 minutes after the termination solution is added.
15. Plotting the standard curve and calculating the concentration. The standard curve is obtained with the OD values as the horizontal ordinate and the concentration of the standards as the vertical coordinate, and a regression equation is obtained. The OD values of sample to be tested are substituted into the equation to get the concentration of the sample to be tested.

### Example 4

### Detecting PDCD5 Protein by Using Antibody Sandwich ELISA Method

PDCD5 protein (Acta Scientiarum Naturalium Universitatis Pekinensis (Medical edition), 2003, 34 (4): 360-363) is doubling diluted from 200 ng/ml into 8 serial dilutions: 200 ng/ml, 100 ng/ml, 50 ng/ml, 25 ng/ml, 12.5 ng/ml, 6.25 ng/ml, 3.125 ng/ml, 1.56 ng/ml, and each of the sample solution is set in 3 wells at a volume of 0.1 ml/well. Three repeating detections are carried out with the ELISA method established in Example 3, and the results are similar, as shown in FIG. 3. There is good concentration-dependent between the reaction of PDCD5 with the antibody, and the detecting range is 1 ng/ml-200 ng/ml.

### Example 5

### Detecting the PDCD5 Protein in the Serum of Healthy Human and Patients with Multiple Sclerosis by Using ELISA Method

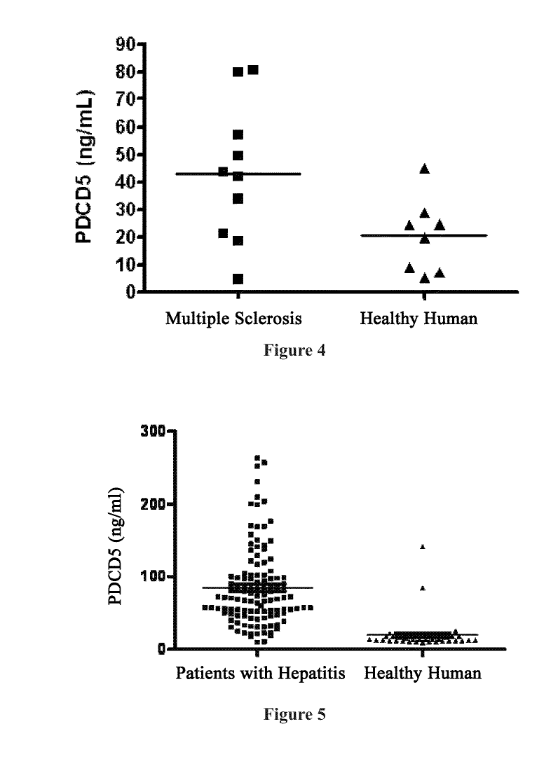

Serum is collected from 8 healthy human (Normal) and 10 patients with multiple sclerosis (multiple sclerosis, MS), and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in FIG. 4. The content of PDCD5 protein in the serum of the 10 patients with multiple sclerosis is 47.39±22.323 ng/ml, and the content of PDCD5 protein in the serum of the 8 healthy human is 20.49±13.32 ng/ml. The content of PDCD5 protein in the serum of patients with multiple sclerosis is significantly higher than that of in the serum of healthy human, and the significance has statistical significance (p<0.01).

### Example 6

### Detecting the PDCD5 Protein in the Serum of Healthy Human and Patients with Hepatitis By Using ELISA Method

Serum is collected from 42 healthy human and 118 patients with hepatitis, and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in FIG. 5. The average content of PDCD5 protein in the serum of the 118 patients with hepatitis is 84.85±54.09 ng/ml, and the average content of PDCD5 protein in the serum of the 42 healthy human is 20.35±12.35 ng/ml. The content of PDCD5 protein in the serum of patients with hepatitis is significantly higher than that of in the serum of healthy human, and the significance has statistical significance (p<0.0001).

### Example 7

### Detecting the PDCD5 Protein in the Serum of Healthy Human and Patients with Influenza A by Using ELISA Method

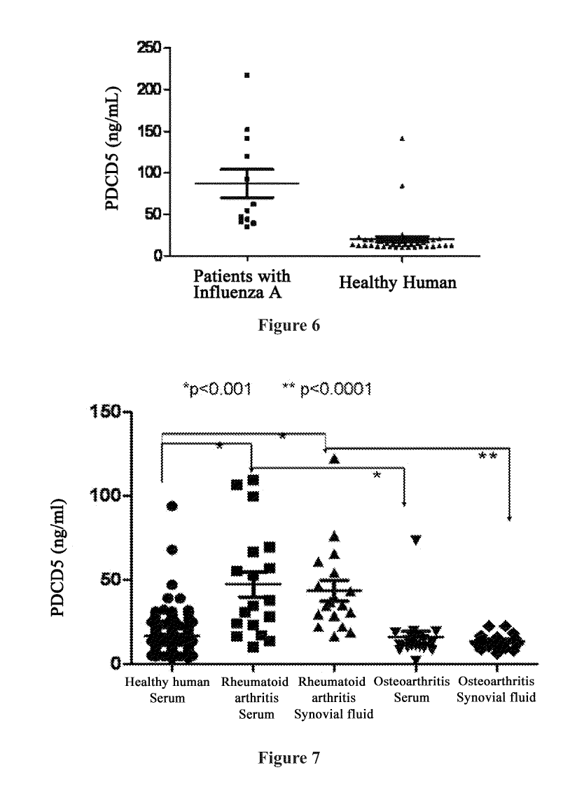

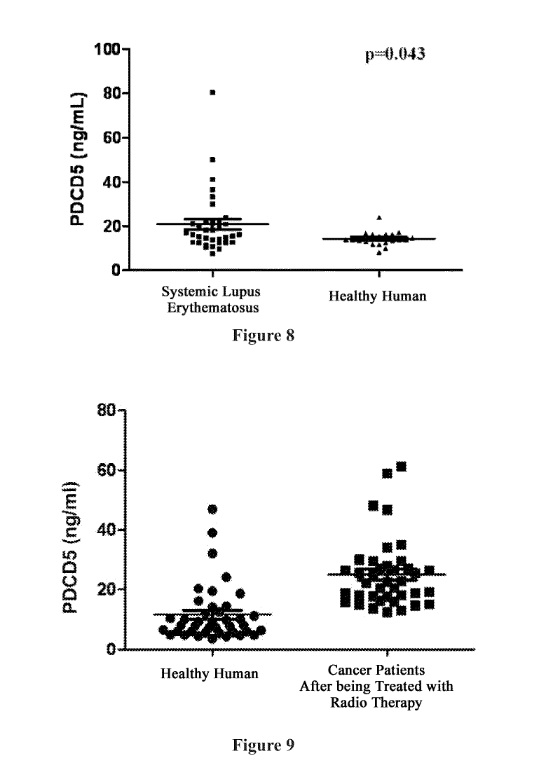

Serum is collected from 42 healthy human and 12 patients with Influenza A, and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in FIG. 6. The average content of PDCD5 protein in the serum of the 12 patients with Influenza A is 87.28±58.92 ng/ml, and the average content of PDCD5 protein in the serum of the 42 healthy human is 20.35±12.35 ng/ml. The content of PDCD5 protein in the serum of patients with Influenza A is significantly higher than that of in the serum of healthy human, and the significance has statistical significance (p<0.0001).

### Example 8

### Detecting the PDCD5 Protein in the Serum and Synovial Fluid of Healthy Human, Patients With Rheumatoid Arthritis and Patients with Osteoarthritis by Using ELISA Method

Serum is collected from 167 healthy human, 20 patients with rheumatoid arthritis and 19 patients with osteoarthritis and the synovial fluid is collected from 20 patients with rheumatoid arthritis and 19 patients with osteoarthritis. Then the collected serum and synovial fluid is diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in FIG. 7. The average content of PDCD5 protein in the serum of the 167 healthy human is 16.55±8.81 ng/ml; the average content of PDCD5 protein in the serum of the 20 patients with rheumatoid arthritis is 47.38±32.14 ng/ml; the average content of PDCD5 protein in the synovial fluid of the 20 patients with rheumatoid arthritis is 43.55±25.75 ng/ml; the average content of PDCD5 protein in the serum of the 19 patients with osteoarthritis is 15.87±15.04 ng/ml; the average content of PDCD5 protein in the synovial fluid of the 19 patients with osteoarthritis is 13.44±4.65 ng/ml. The level of PDCD5 protein in the serum of patients with rheumatoid arthritis is significantly higher than that of in the serum of healthy human, and the significance has statistical significance (* p<0.001). Meanwhile, the level of PDCD5 protein in the synovial fluid of patients with rheumatoid arthritis is significantly higher than that of in the synovial fluid of healthy human, and the significance has statistical significance (* p<0.001). The level of PDCD5 in the serum of patients with osteoarthritis is not significantly different from that of in the serum of healthy human, while the level of PDCD5 in the serum of patients with osteoarthritis is lower than that of in the serum of patients with rheumatoid arthritis, and the difference has statistical significance (* p<0.001); at the same time, the level of PDCD5 in the synovial fluid of patients with osteoarthritis is lower than that of in the synovial fluid of patients with rheumatoid arthritis, and the difference has statistical significance (* p<0.0001).

### Example 9

### Detecting the PDCD5 Protein in the Serum of Healthy Human and Patients with Systemic Lupus Erythematosus by Using ELISA Method

Serum is collected from 20 healthy human and 35 patients with systemic lupus erythematosus, and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in FIG. 6. The average content of PDCD5 protein in the serum of the 35 patients with systemic lupus erythematosus is 20.84±13.89 ng/ml, and the average content of PDCD5 protein in the serum of the 20 healthy human is 14.28±3.23 ng/ml. The content of PDCD5 protein in the serum of patients with systemic lupus erythematosus is significantly higher than that of in the serum of healthy human, and the significance has statistical significance (p<0.05).

### Example 10

### Detection of the PDCD5 Protein in the Amniotic Fluid of Pregnant Woman and Maternity Patient by Using Antibody Sandwich ELISA Method

80 samples of amniotic fluid are collected from pregnant woman and maternity patient at different stages and the operation is conducted according to the ELISA method as established in Example 3, and then the contents of soluble PDCD5 protein in the samples are calculated by using standard curves. A summary of the results is shown in Table 1, and the content of PDCD5 in the 80 samples of amniotic fluid is 3.561±1.068 ng/ml (n=80).

TABLE 1
Results of the detection for PDCD5 protein in the amniotic
fluid of pregnant woman and maternity patient by using
antibody sandwich ELISA method. (ng/ml)

| | PDCD5 |
|---|---|
| Sample | Protein |
| 1 | 2.007 |
| 2 | 1.961 |
| 3 | 2.111 |
| 4 | 2.166 |
| 5 | 2.145 |
| 6 | 2.076 |
| 7 | 2.101 |
| 8 | 1.955 |
| 9 | 1.969 |
| 10 | 2.096 |
| 11 | 2.2801 |
| 12 | 1.975 |
| 13 | 1.955 |
| 14 | 2.103 |
| 15 | 1.985 |
| 16 | 1.971 |
| 17 | 1.983 |
| 18 | 2.163 |
| 19 | 2.166 |
| 20 | 2.079 |
| 21 | 2.185 |
| 22 | 2.208 |
| 23 | 2.191 |
| 24 | 1.956 |
| 25 | 2.105 |
| 26 | 2.105 |
| 27 | 2.1058 |
| 28 | 2.105 |
| 29 | 2.105 |
| 30 | 2.105 |
| 31 | 2.105 |
| 32 | 2.105 |
| 33 | 1.956 |
| 34 | 2.052 |
| 35 | 2.391 |
| 36 | 1.955 |
| 37 | 2.098 |
| 38 | 2.465 |
| 39 | 2.382 |
| 40 | 1.996 |
| 41 | 3.083 |
| 42 | 2.678 |
| 43 | 4.347 |
| 44 | 5.857 |
| 45 | 2.858 |
| 46 | 6.375 |
| 47 | 3.673 |
| 48 | 4.393 |
| 49 | 4.692 |
| 50 | 5.091 |
| 51 | 5.989 |
| 52 | 5.244 |
| 53 | 5.005 |
| 54 | 6.818 |
| 55 | 4.486 |
| 56 | 5.402 |
| 57 | 4.628 |
| 58 | 5.005 |
| 59 | 5.581 |
| 60 | 6.199 |
| 61 | 6.838 |
| 62 | 5.421 |
| 63 | 5.636 |
| 64 | 6.534 |
| 65 | 4.971 |
| 66 | 5.473 |
| 67 | 3.978 |
| 68 | 3.851 |
| 69 | 5.262 |
| 70 | 5.691 |
| 71 | 5.857 |
| 72 | 5.709 |
| 73 | 5.473 |
| 74 | 4.471 |
| 75 | 4.955 |
| 76 | 4.888 |
| 77 | 6.141 |
| 78 | 5.709 |
| 79 | 2.587 |
| 80 | 5.473 |

### Example 11

### Detecting the PDCD5 Protein in the Serum of Healthy Human and Patients with Myeloma by Using ELISA Method

Serum is collected from 12 healthy human and 63 patients with myeloma, and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in Table 2. The content of PDCD5 protein in the serum of the 63 patients with myeloma is 17.19±1.56 ng/ml, and the content of PDCD5 protein in the serum of the 12 healthy human is 19.30±0.39 ng/ml. The content of PDCD5 protein in the serum of patients with myeloma is significantly lower than that of in the serum of healthy human, and the significance has statistical significance (p<0.05).

TABLE 2
Results of the detection for PDCD5 protein in the serum of
healthy human and patients with myeloma by using antibody
sandwich ELISA method.

| | | | | | | | | Healthy | |
|---|---|---|---|---|---|---|---|---|---|
| | | | | | | | | human | |
| | Patients with myeloma (n = 63) | | | | | | | (n = 12) | |
| | 1 | 2 | 3 | 4 | 5 | 6 | 7 | 8 | 9 |
| 1 | 17.18 | 17.41 | 18.65 | 17.01 | 7.18 | 16.83 | 17.53 | 19.08 | 19.08 |
| 2 | 18.03 | 17.52 | 17.01 | 17.40 | 12.41 | 17.18 | 16.12 | 19.34 | 19.34 |
| 3 | 17.66 | 18.22 | 16.89 | 16.54 | 16.91 | 17.54 | 18.74 | 19.47 | 19.15 |
| 4 | 16.63 | 18.64 | 16.31 | 17.05 | 17.41 | 17.58 | 17.29 | 19.29 | |
| 5 | 18.23 | 18.12 | 17.12 | 17.08 | 16.34 | 17.23 | 16.62 | 17.64 | |
| 6 | 18.05 | 17.892 | 17.15 | 17.18 | 16.65 | 17.13 | 17.13 | 17.63 | |
| 7 | 18.81 | 18.332 | 17.27 | 16.86 | 17.67 | 17.19 | 17.55 | 18.39 | |
| 8 | 17.25 | 18.512 | 16.89 | 17.43 | 17.16 | 17.82 | 16.63 | 18.19 | |
| 9 | 18.12 | 18.242 | 17.01 | 17.47 | 17.62 | 18.15 | 18.28 | 18.99 | |

### Example 12

### Detecting the PDCD5 Protein in the Serum of Healthy Human and Patients with Chronic Myelogenous Leukemia by Using ELISA Method

Serum is collected from 12 healthy human and 18 patients with chronic myelogenous leukemia, and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in Table 3. The content of PDCD5 protein in the serum of the 18 patients with chronic myelogenous leukemia is 17.69±1.34 ng/ml, and the content of PDCD5 protein in the serum of the 12 healthy human is 19.30±0.39 ng/ml. The content of PDCD5 protein in the serum of patients with chronic myelogenous leukemia is significantly lower than that of in the serum of healthy human, and the significance has statistical significance (p<0.05).

TABLE 3
Results of the detection for PDCD5 protein in the serum of healthy
human and patients with chronic myelogenous leukemia by using
antibody sandwich ELISA method.

| | |
|---|---|
| Chronic myelogenous | Healthy human |
| leukemia (n = 18) | (n = 12) |

| | 1 | 2 | 3 | 4 | 5 |
|---|---|---|---|---|---|
| 1 | 17.31 | 17.82 | 17.58 | 19.08 | 19.08 |
| 2 | 18.40 | 18.06 | 17.76 | 19.34 | 19.34 |
| 3 | 18.39 | 17.47 | 18.06 | 19.47 | 19.15 |
| 4 | 17.18 | 17.70 | 17.81 | 19.29 | 18.39 |
| 5 | 18.11 | 18.43 | 18.45 | 17.64 | 18.19 |
| 6 | 18.07 | 19.21 | 12.68 | 17.63 | 18.99 |

### Example 13

### Detecting the PDCD5 Protein in the Serum of Healthy Human and Cancer Patients after Treated with Radio Therapy by Using ELISA Method

Serum is collected from 40 healthy human and 39 cancer patients after treated with radio therapy, and diluted with sample diluting solution at a ratio of 1:10, and then the operation is conducted according to the ELISA method as established in Example 3. The contents of soluble PDCD5 protein in the samples are calculated by using standard curves, and then the results are statistically treated by t-test. The results are shown in FIG. 9. The content of PDCD5 protein in the serum of the 39 cancer patients after treated with radio therapy is 25.11±11.61 ng/ml, and the content of PDCD5 protein in the serum of the 40 healthy human is 11.62±9.52 ng/ml. The content of PDCD5 protein in the serum of cancer patients after treated with radio therapy is significantly higher than that of in the serum of healthy human, and the significance has statistical significance (p<0.0001).

1. An ELISA method for detecting soluble PDCD5 protein, comprising the following steps:
(1) Contacting a sample to be tested with a solid carrier loaded with a first antibody of PDCD5 protein;
(2) Adding a second antibody of PDCD5 protein, which is capable of binding to a detecting label;
(3) Adding the detecting label and detecting the bound detecting label.

2. The method of claim 1, characterized in that the sample to be tested is serum, plasma, urine, pleural-peritoneal effusion, synovial fluid, cerebrospinal fluid, amniotic fluid, supernatant of a cell culture or cell lysate.

3. The method of claim 1, characterized in that the method further comprises the following steps: repeating steps (1) to (3) except for replacing a series of solutions comprising soluble PDCD5 protein with known concentration with the sample to be tested, and plotting standard curves; repeating steps (1) to (3) with sample to be tested; determining the concentration of soluble PDCD5 protein in the sample to be tested in accordance with the standard curves.

4. The method of claim 1, characterized in that the first antibody of PDCD5 protein is a monoclonal antibody of PDCD5 protein, such as a mouse monoclonal antibody against human PDCD5 protein.

5. The method of claim 1, characterized in that the second antibody of PDCD5 protein is a polyclonal antibody of PDCD5 protein, such as a rabbit polyclonal antibody against human PDCD5 protein.

6. The method of claim 1, characterized in that the detecting label is enzyme, fluoresce or isotope.

7. An ELISA kit for detecting soluble PDCD5 protein, which comprises:
a first antibody of PDCD5 protein for capturing the soluble PDCD5 protein in the sample to be tested;
a second antibody of PDCD5 protein which is capable of binding to a detecting label;
a series of solutions comprising soluble PDCD5 protein with known concentrations;
a detecting label;
a tool for detecting the detecting label.

8. The ELISA kit of claim 7, characterized in that the first antibody of PDCD5 protein is a monoclonal antibody of PDCD5 protein, such as a mouse monoclonal antibody against human PDCD5 protein.

9. The ELISA kit of claim 7, characterized in that the second antibody of PDCD5 protein is a polyclonal antibody of PDCD5 protein, such as a rabbit polyclonal antibody against human PDCD5 protein.

10. The ELISA kit of claim 7, characterized in that the detecting label is enzyme, fluoresce or isotope.

11. The ELISA kit of claim 7, characterized in that the tool for measuring the detecting label is a tool for detecting enzyme, fluoresce or isotope.
